(19) United States
(12) Patent Application Publication
(10) Pub. No.: US 2012/0107425 A1
(43) Pub. Date: May 3, 2012

(54) USE OF RHODIOLA CRENULATA EXTRACT VIA THE TOPICAL ROUTE

(76) Inventors: Eric Perrier, Les Cotes D'Arey (FR); Sébastien Bonnet, Saint Jean de Niost (FR); Delphine Rival, Ternay (FR); Patrice Andre, Neuville Aux Bois (FR); Nancy About, Neudon (FR)

(21) Appl. No.: 13/169,601

(22) Filed: Jun. 27, 2011

Related U.S. Application Data
(63) Continuation of application No. 10/842,904, filed on May 10, 2004, now abandoned.

(30) Foreign Application Priority Data
Aug. 28, 2003 (FR) 0310249

(51) Int. Cl. A61K 36/41; A61P 17/10; A61P 17/00

(52) U.S. Cl. 424/725

(57) ABSTRACT

The invention relates to the use of a Rhodiola crenulata extract, via the topical route, for increasing the energy metabolism of the cutaneous tissues of a subject.

The invention relates notably to topical compositions which comprise an active agent for increasing the energy metabolism of the cutaneous tissues of a subject, said active agent being a Rhodiola crenulata extract.

The present invention enables making a cosmetic care such as: fighting against, or slowing down or preventing the loosening of the cutaneous tissue, increasing the plasticity of the cutaneous tissue, exerting an anti-wrinkle effect or slowing down or preventing the appearance of wrinkles, fighting against, slowing down or preventing the appearance of acne, exerting a depigmenting effect of the cutaneous tissue, in particular of the skin, and promoting desquamation of the cutaneous tissue (or “peeling”).

The invention relates to the use of a Rhodiola crenulata extract, via the topical route, for increasing the energy metabolism of the cutaneous tissues of a subject.

### STATE OF THE ART

It is known that this family of plants, which is also called Rhodiola, grows in altitude, in an environment which is unfavorable even hostile, and that it possesses adaptogenic properties. The extracts of Rhodiola have been used via the oral route, in the form of alcoholic drinks for multiple applications: effects on the cardiovascular system, effects on the memory, anti-histaminic effects, etc. . . . , without the effects being studied well from a biological mechanistic point of view. Furthermore, the effects have been described mainly about Rhodiola rosea, Rhodiola kirilowii and Rhodiola sachalinensis, since Rhodiola crenulata is a plant which is relatively difficult to find, it only grows on the high Tibetan plateaux, and, due to this, has been the subject of only very few studies up to now.

Furthermore, cosmetic applications without a directly demonstrated biological activity have been described for Rhodiola rosea (JP 2001048768) or for Rhodiola sachalinensis (CN 1306816).

To conclude, Rhodiola crenulata extracts have been described for uses via the oral route for pharmaceutical applications, in particular for increasing the levels of testosterone and of oestradiol in the blood, or for promoting the transport of oxygen in the blood (US 2002/0127285=U.S. Pat. No. 6,399,116). It is also described in this document that the efficient transport of oxygen enables reducing muscular fatigue by increasing the levels of ATP which is present in the muscle fibers. This enables muscular fatigue to be reduced. However, the studies are restricted to this field, and the person skilled in the art cannot draw any teaching as to an eventual cosmetic or dermopharmaceutical use of the extracts of Rhodiola crenulata for increasing the cell metabolism of the cutaneous tissues.

At present, much research has been done, notably by specialists in cutaneous tissues, in order to demonstrate solutions which enable notably alleviating the loosening of the cutaneous tissue and the appearance of wrinkles, by using methods which are in general quite aggressive, of the cutaneous tissue “peeling” type, so as to make surface wrinkles disappear and to fight against its loosening.

Much research has also taken place in the field relating to the fight against acne.

In the same way, much research has taken place in order to alleviate various problems of the cutaneous tissue, in particular of the skin, such as the presence of various areas of pigmentation.

Despite the extent of this research, a deficiency in the solutions brought about to these various problems does exist at present.

Furthermore, the users of cosmetic products are sensitive to the plant origin of the compositions that are proposed to them and thus prefer using compositions which contain plant extracts.

### AIMS OF THE INVENTION

The invention thus relates to alleviating the deficiencies of the state of the art by solving the various technical problems set forth above and below.

A main aim of the invention is to solve the novel technical problem which consists of providing novel topical compositions which enable increasing the energy metabolism of the cutaneous tissues of a subject, notably of the skin, with the particular view to reducing, slowing down or preventing the appearance of wrinkles or exerting an anti-wrinkle effect, and/or to increasing the plasticity of the cutaneous tissue, and/or to fighting against, slowing down or preventing the loosening of the cutaneous tissue, and/or to fighting against, slowing down or preventing the appearance of acne, and/or to exerting a depigmenting effect of the cutaneous tissue, in particular of the skin, and/or to promoting desquamation of the cutaneous tissue (or “peeling”).

### DESCRIPTION OF THE INVENTION

The whole of these technical problems is solved for the first time in a satisfactory manner by the invention, notably on an industrial scale, and in particular on a cosmetic scale.

According to a first aspect, the invention relates to the use of a Rhodiola crenulata extract for the manufacture of a topical composition for increasing the energy metabolism of the cutaneous tissues of a subject.

The energy metabolism of the cutaneous tissues is represented advantageously by the determination of cytosolic ATP.

Advantageously, the energy metabolism of the cutaneous tissue can also be measured in situ, e.g. by NMR (Nuclear Magnetic Resonance) spectroscopy, particularly of phosphorus 31.

In particular, the variations of the energy metabolism of the skin can be determined by phosphorus 31 NMR spectroscopy by virtue of the measurement of the relative concentrations of the principle phosphorylated metabolites of the skin: inorganic phosphate (Pi), phosphocreatine (PCr) and adenosine triphosphate (ATP).

The ATP molecules provide the necessary energy to the cells and to the tissues. Phosphocreatine is a molecule which constitutes an energy reserve which is immediately available, and whose role is to reconstitute the ATP reserves consumed during a period of cellular ischemia by providing a phosphate group to the ADP molecules, according to the reaction which is catalyzed by creatine phosphokinase:

ADP+PCr [symbol] ATP+Cr

The evolution of the PCr/Pi, ATP/Pi, PCr/ATP and PCr/Ptotal ratios are a reflection of the energy status of a tissue.

The invention itself is not limited to the particular analysis which enables demonstrating the evaluation of the energy metabolism, but the methods which are indicated above are advantageously used in the invention and in particular provide an efficient rapidity and precision of analysis, in order to reach the goal sought after.

According to an embodiment, said cutaneous tissue is the skin. Preferably, said cutaneous tissue is the epidermis and/or the dermis, in particular of the human being.

Advantageously, this use enables increasing the energy metabolism of the keratinocytes and/or of the fibroblasts.

According to an embodiment, the subject is a mammal, particularly the human being.

Advantageously, the topical composition comprises between 0.01% and 10% (w/w) of extract.

According to another embodiment, the extract contains crenulatine, or at least one of its salts or at least one of its derivatives, such as its acid or ester derivatives.

According to yet another embodiment, the Rhodiola crenulata extract is prepared by extraction, preferably of the roots, and/or of the low stem of the plant, by a solvent, followed optionally by a fractioning step enabling the active fraction(s) to be isolated. The solvent can be polar or non-polar. Said solvent is preferably selected from the group consisting of pentane, decane, cyclohexane, hexane, petroleum ether, monochloromethane, dichloromethane, chloroform, isopropanol, propanol, ethyl acetate, ethanol, methanol, acetone, butylene glycol, propylene glycol, pentylene glycol, glycerol, water, and any mixture of at least two of these solvents, particularly water-alcoholic or water-glycolic mixtures.

Advantageously, the extract is purified.

Advantageously, this use enables fighting against, or slowing down or preventing the loosening of the cutaneous tissue, and/or increasing the plasticity of the cutaneous tissue, and/or exerting an anti-wrinkle effect, and/or fighting against, slowing down or preventing the appearance of acne, and/or exerting a depigmenting effect of the cutaneous tissue, in particular of the skin, and/or promoting desquamation of the cutaneous tissue (or “peeling”).

In particular, the present invention enables increasing the capacity of the cells of the cutaneous tissue to multiply, i.e. increasing the cellular proliferation of these cells, in particular of the keratinocytes and of the fibroblasts.

Advantageously, this increase of the cellular proliferation in the epidermis enables a more rapid renewal of the epidermis.

Advantageously, this more rapid renewal of the epidermis enables modifying the amount of melanin present in the epidermis, and this thus enables modifying the pigmentation of the tissue and exerting a depigmenting effect of the cutaneous tissue. This effect is used notably for lightening the skin of a human being or of an animal.

Advantageously, this increase of the cellular proliferation enables thickening the epidermis so as to restore the role of barrier effect of the epidermis, e.g. on an aged skin, which is generally very fine.

Advantageously, this increase of the cell proliferation enables promoting desquamation, e.g. in cases of dry skin, which, by definition, have difficulty in desquaming.

Advantageously, the increase of the cellular proliferation, in particular of the fibroblasts in the dermis, enables densifying this dermis and thus restoring the plasticity of it and/or its firmness notably so as to exert an anti-wrinkle effect or to fight against, to slow down or to prevent the appearance of wrinkles.

According to a second aspect, the invention relates to a topical composition which comprises an active agent for increasing the energy metabolism of the cutaneous tissue on at least the part on which said composition is applied, said active agent being a Rhodiola crenulata extract, as defined above.

Advantageously, the extract is in a mixture with an excipient which is acceptable via the topical route, particularly a cosmetically or dermatologically acceptable excipient.

For these compositions, the excipient contains for example at least one compound selected from the group consisting of preservatives, emollients, emulsifiers, surfactants, moisturizers, thickeners, conditioners, matifying agents, stabilizers, antioxidants, texture agents, brightening agents, filmogenic agents, solubilizers, pigments, dyes, perfumes and solar filters. These excipients are preferably selected from the group consisting of amino acids and their derivatives, polyglycerols, esters, polymers and derivatives of cellulose, lanolin derivatives, phospholipids, lactoferrins, lactoperoxidases, sucrose-based stabilizers, E vitamins and its derivatives, natural and synthetic waxes, plant oils, triglycerides, insaponifiables, phytosterols, plant esters, silicones and its derivatives, protein hydrolyzates, jojoba oil and its derivatives, lipo/hydrosoluble esters, betaines, aminoxides, plant extracts, esters of sucrose, titanium dioxides, glycines, and parabens, and more preferably from the group consisting of butylene glycol, steareth-2, steareth-21, glycol-15 stearyl ether, cetearyl alcohol, phenoxyethanol, methylparaben, ethylparaben, propylparaben, butylparaben, butylene glycol, natural tocopherols, glycerol, sodium dihydroxycetyl, isopropyl hydroxycetyl ether, glycol stearate, triisononaoine, octyl cocoate, polyacrylamide, isoparaffin, laureth-7, a carbomer, propylene glycol, glycerol, bisabolol, dimethicone, sodium hydroxide, PEG 30-dipolyhydroxysterate, capric/caprylic triglycerides, cetearyl octanoate, dibutyl adipate, grape seed oil, jojoba oil, magnesium sulfate, EDTA, a cyclomethicone, xanthan gum, citric acid, sodium lauryl sulfate, mineral waxes and oils, isostearyl isostearate, propylene glycol dipelargonate, propylene glycol isostearate, PEG 8 Beeswax, hydrogenated palm tree heart oil glycerides, hydrogenated palm oil glycerides, lanolin oil, sesame oil, cetyl lactate, lanolin alcohol, castor oil, titanium dioxide, lactose, sucrose, low density polyethylene, and an isotonic saline solution.

Advantageously, the compositions cited above are formulated in a form selected from the group consisting of a solution, which is aqueous or oily, an aqueous cream or gel or an oily gel, notably in a pot or in a tube, notably a shower gel, a shampoo; a milk; an emulsion, a microemulsion or a nanoemulsion, notably an oil-in-water or water-in-oil or multiple or silicone-containing microemulsion or nanoemulsion; a lotion, notably in a glass bottle, a plastic bottle or in a measure bottle or in an aerosol; an ampoule; a liquid soap; a dermatological bar; an ointment; a foam; an anhydrous product, preferably a liquid, pasty or solid anhydrous product, e.g. in the form of a stick, notably in the form of a lipstick.

According to a third aspect, the invention relates to a method of preparing said Rhodiola crenulata extract, which comprises reducing in powder form at least one part of the plant Rhodiola crenulata, preferably the roots and/or the low stem, e.g. by grinding.

The whole of the methods known by the person skilled in the art can be employed for carrying out the extraction.

Particularly advantageously, the powder is subjected to an extraction in using a solvent or a mixture of solvents, and the product thus obtained is optionally subjected to steps of fractionation enabling isolating the active fraction(s). The active fraction(s) is (are) advantageously filtered on an adequate filter in order to obtain a solid matter containing the substance which is active with respect to the purpose sought after. The powder is preferably left to soak in a solvent or mixture of solvents, e.g. at ambient temperature or under reflux of the solvent. Said solvent is advantageously selected from those which are described above.

The active substance obtained after filtration is advantageously mixed with at least one excipient which is topically acceptable, as defined above.

The person skilled in the art will advantageously prepare the formulation, which is adequate for the purpose sought after, through this technical competences.

According to a fourth aspect, the invention relates to a method of cosmetic care, characterized in that it comprises topically applying, on at least one area of the cutaneous tissue of a subject, said composition comprising a Rhodiola crenulata extract, for increasing the energy metabolism of at least the area of the cutaneous tissue of the subject.

Advantageously, the method of cosmetic care enables exerting a care selected from the group consisting of fighting against, or slowing down or preventing the loosening of the cutaneous tissue, increasing the plasticity of the cutaneous tissue, exerting an anti-wrinkle effect or slowing down or preventing the appearance of wrinkles, fighting against, slowing down or preventing the appearance of acne, exerting a depigmenting effect of the cutaneous tissue, in particular of the skin, and promoting desquamation of the cutaneous tissue (or “peeling”).

According to a particular embodiment, the method of cosmetic care enables increasing the capacity of the cells of the cutaneous tissue to multiply, i.e. to increase the cellular proliferation of these cells, in particular of the keratinocytes and/or of the fibroblasts, and therefore to obtain the effects described above in particular.

Advantageously, the cosmetic care is made on a subject in need thereof, particularly by carrying out a prior step of selecting said subjects with respect to a population.

Advantageously, said part of the cutaneous tissue on which the composition is applied is selected as a function of the care to be made.

According to a fifth aspect, the invention relates to a method of therapeutic treatment which comprises topically applying, on at least one area of the cutaneous tissue of a subject, a therapeutically effective amount of said composition comprising a Rhodiola crenulata extract, thus enabling increasing the energy metabolism of at least said area of the cutaneous tissue of the subject.

Advantageously, this method of treatment enables making a treatment selected from the group consisting of fighting against, or slowing down or preventing the loosening of the cutaneous tissue, increasing the plasticity of the cutaneous tissue, exerting an anti-wrinkle effect or slowing down or preventing the appearance of wrinkles, fighting against, slowing down or preventing the appearance of acne, exerting a depigmenting effect of the cutaneous tissue, in particular of the skin, and promoting desquamation of the cutaneous tissue (or “peeling”).

According to a particular embodiment, the method of therapeutic treatment enables increasing the capacity of the cells of the cutaneous tissue to multiply, i.e. to increase the cellular proliferation of these cells, in particular of the keratinocytes and/or of the fibroblasts, and therefore to obtain the effects described above in particular.

Advantageously, this method of treatment is made on a subject in need of this treatment, particularly by carrying out a prior step of selecting said subjects with respect to a population.

Advantageously, said part of the cutaneous tissue on which the composition is applied is selected as a function of the treatment to be made.

In general, the tissue is preferably the skin or the mucous membranes of the subject.

The topical composition of the present invention enables in particular obtaining a local effect of increase of the metabolism of the cutaneous tissue at the area of application.

Other aims, features and advantages of the invention will appear clearly to the person skilled in the art upon reading the explanatory description which makes reference to the following Examples, which are given solely as an illustration and which in no way limit the scope of the invention.

The Examples make up an integral part of the present invention and any feature appearing novel with respect to any prior state of the art from the description taken in its entirety, including the Examples, makes up an integral part of the invention in its function and in its generality.

Thus, every Example is of general scope.

Furthermore, in the Examples, all the percentages are given by weight, unless indicated otherwise, and the temperature is expressed in degrees Celsius unless indicated otherwise, and the pressure is atmospheric pressure, unless indicated otherwise.

### EXAMPLES

### Example 1

### Preparation of a Rhodiola crenulata Extract

A Rhodiola crenulata extract was made from roots which were cut, and then ground into a powder. The powder obtained was mixed with ethanol in order to obtain a 10% (w/w) solution in ethanol. This solution was brought to reflux. The extraction was carried out for 1 hour, and the solution was then brought to ambient temperature and was filtered, and the ethanol was removed. The result was dissolved at 5% (w/w) in a water/glycol 75/25 (w/w) mixture, and was then ultrafiltered on a ceramic filter having various cut-off thresholds, and was then finally filtered at 0.45 μm.

0.1% of methyl paraben, used as preservative, was then added last in the presence or not of a xanthan gel.

### Example 2

### Preparation of a Rhodiola crenulata Extract

A Rhodiola crenulata extract was made from roots which were cut, and then ground into a powder. The powder obtained was mixed with cyclohexane in order to obtain a 10% (w/w) solution in cyclohexane. This solution was brought to reflux. The extraction was carried out for 24 hours, and the solution was then brought to ambient temperature and was filtered, and the cyclohexane was removed. The result was dissolved at 5% (w/w) in a water/glycol 75/25 (w/w) mixture, and was then ultrafiltered on ceramic filters having various cut-off thresholds, and was then finally filtered at 0.45 μm.

0.1% of methyl paraben, used as preservative, was then added last in the presence or not of a carbomer gel.

### Example 3

### Preparation of a Rhodiola crenulata Extract

A Rhodiola crenulata extract was made from roots which were cut, and then ground into a powder. The powder obtained was mixed in a solvent made up of 75% water and 25% butylene glycol, in order to obtain a 10% (w/w) solution in the solvent. Soaking was allowed for 1 night at 45° C., and the solution was then ultrafiltered on ceramic filters having various cut-off thresholds, and was then finally filtered at 0.45 μm.

0.1% of methyl paraben, used as preservative, was then added last in the presence or not of a cellulose gel.

### Example 4

### Preparation of a Rhodiola crenulata Extract

A Rhodiola crenulata extract was made from roots which were cut, and then ground into a powder. The powder obtained was mixed with a solvent made up of 75% water and 25% butylene glycol, in order to obtain a 1% (w/w) solution in the solvent. Soaking was allowed for 1 night at 4° C., and the solution was then ultrafiltered on ceramic filters having various cut-off thresholds, and was then finally filtered at 0.45 μm.

0.1% of methyl paraben, used as preservative, was then added last in the presence or not of a xanthan gel.

### Example 5

### Cytotoxicity Study

Study of the cytotoxicity of the Rhodiola crenulata extract prepared according to Example 4 on keratinocytes (determination with para-nitrophenyl phosphate, PNPP).

Normal human keratinocytes were extracted from an abdominal biopsy. They were sown in 96-well culture plates. Once the confluence was attained, the cells were incubated in a culture medium alone (control) or containing 0.01%, 0.1%, 1%, 3%, 5% and 10% (w/w) of the Rhodiola crenulata extract prepared according to Example 4, for 24 hours at 37° C. in an atmosphere containing 5% CO₂.

At the end of the incubation period, the cell viability was evaluated by a test with PNPP. This test enables evaluating the activity of the intracellular acid phosphatases, which is directly proportional to the number of viable cells.

200 μl of 5 mM PNPP solution (Sigma) in demineralized water containing 0.1% of triton X100 (Sigma) and 0.1M of sodium acetate (Sigma) at pH 5 were placed in each culture well. An incubation at 37° C. for 2 hours was made, and the reaction was then stopped by the addition of 1N NaOH (Merck). A reading of the absorbance is made at 405 nm and the results are expressed as a percentage of viable cells with respect to control (without active).

TABLE I

| Concentration of | | |
|---|---|---|
| Rhodiola crenulata | Percentage viability | Standard |
| extract | on keratinocytes | deviation |
| 0.01% | 95.9 | 3.3 |
| 0.1% | 92.4 | 5.5 |
| 1% | 92.2 | 4.8 |
| 3% | 84.5 | 8 |
| 5% | 79.4 | 5.9 |
| 10% | 55.7 | 5.3 |

### Example 6

### Evaluation of the Energy Metabolism on Human Keratinocytes in Culture of the Extract Obtained in Example 4

### Measurement of Cellular ATP Levels

Normal human keratinocytes are sown at the rate of 15.10³ cells per well in 96-well microplates. After 72 hours of incubation at 37° C., the culture medium is removed and replaced by a medium containing the active at various concentrations.

The cells are incubated for 3 consecutive days, and the cells are then rinsed with PBS buffer. The determination of the cytosolic ATP is determined by a bioluminescence technique, according to the reaction:

Luciferin+O₂+Luciferase+ATP→Oxyluciferin+AMP+PPi+CO₂+Luciferase+Photons

After rinsing with PBS, the keratinocytes are incubated for 5 minutes in a permeabilization buffer containing digitonin. A dilute solution of AMR (ATP monitoring reagent, Roche Diagnostic) is injected directly into the microplates and the intensity of light emitted (RLU total) is measured by a luminometer (Luminoscan, Labsystem). After a first reading, the microplate wells receive a solution of hexokinase (Sigma) (system using ATP). After 10 minutes of incubation, a second measurement of the light is made (RLU0) enabling the part of the light linked to molecules other than ATP to be subtracted:

RLU_{ATP}=RLU_{Total}−RLU₀

The level of cytosolic ATP is determined from a standard range realized with standard concentrations of ATP.

In parallel with the measurement of the cytosolic ATP, a determination of the cell proteins is made on a second microplate, by a test with Coomassie blue (BioRad Protein Assay).

### Results:

The results (ATP levels) are expressed in pmoles of ATP/μg of proteins. The Table below groups the average values (N=4).

TABLE II

| | | Standard | | ATP |
|---|---|---|---|---|
| | Average | deviation | Stat* | (%) |
| control | 47.86 | 4.77 | n.s. | 100% |
| 0.1% Rhodiola crenulata | 52.68 | 1.72 | n.s. | 110% |
| extract | | | | |
| 0.5% Rhodiola crenulata | 54.04 | 3.44 | n.s. | 113% |
| extract | | | | |
| 1% Rhodiola crenulata | 62.18 | 2.68 | p < 0.01 | 130% |
| extract | | | | |
| 2% Rhodiola crenulata | 76.16 | 2.19 | P < 0.01 | 159% |
| extract | | | | |

Stat*: Student T test

No modification of the cell growth is observed on the batches treated with the active Rhodiola crenulata extract. The levels of cell proteins are in fact identical to that recorded for the control batch (without active).

On the contrary, a slight decrease (11%) of the level of proteins is recorded on cells treated with the 2% (w/w) Rhodiola crenulata extract.

A dose-dependent increase of the ATP levels is recorded on treated cells; the differences recorded on the tests with the 1% and 2% Rhodiola crenulata extract are statistically significant (Student T test).

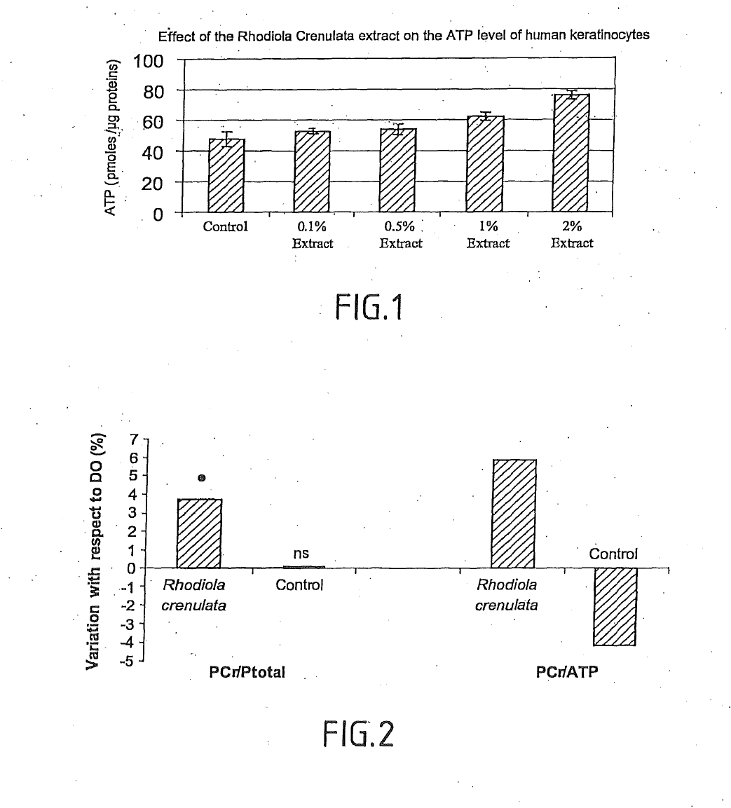

FIG. 1 represents the level of ATP in human keratinocytes as a function of various concentrations of the Rhodiola crenulata extract.

### Conclusion:

Under the experimental conditions described above, the Rhodiola crenulata extract is capable of stimulating the energy metabolism of the human keratinocytes.

A dose-dependent increase of the level of cytosolic ATP was in fact recorded within the range of the concentrations tested.

At the concentration of 2% (w/w), the Rhodiola crenulata extract is capable of inducing an increase of 59% of the level of ATP of the keratinocytes.

### Example 7

### Improvement of the Energy Metabolism of the Cutaneous Tissue

The in situ measurement of the energy metabolism of the cutaneous tissue was made using NMR (Nuclear Magnetic Resonance) spectroscopy. The aim of this study was to evaluate the effect of a formulation containing 3% (w/w) of the Rhodiola crenulata extract on the energy metabolism of the skin, in an in vivo test making use of healthy volunteers. 16 healthy volunteers aged over 18 were included in this study. The 16 healthy volunteers applied a formulation containing 3% (w/w) of the Rhodiola crenulata extract twice daily for 28 days on one of their forearms. Over the same period, 8 of these volunteers applied a placebo formulation on their other forearm, while the other half of the panel applied no treatment on the second forearm (“control” forearms).

Before and after 28 days of twice-daily applications of the formulation to be tested, the energy metabolism of the skin of both the forearms of the volunteers was analyzed by NMR spectroscopy.

The variations of the energy metabolism of the skin can be determined by 31 phosphorus NMR spectroscopy by virtue of the measurement of the relative concentrations of the main phosphorylated metabolites of the skin: inorganic phosphate (Pi), phosphocreatine (PCr) and adenosine triphosphate (ATP).

The ATP molecules provide the energy necessary for the cells and for the tissues. Phosphocreatine is a molecule which constitutes an energy reserve which is immediately available, and whose role is to reconstitute the ATP reserves consumed during a period of cellular ischemia by providing a phosphate group to the ADP molecules, according to the reaction which is catalyzed by creatine phosphokinase:

ADP+PCr [symbol] ATP+Cr

The evolution of the PCr/Pi, ATP/Pi, PCr/ATP and PCr/Ptotal ratios are a reflection of the energy status of a tissue.

The results presented in the Figure below show that the formulation containing the Rhodiola crenulata extract was capable, after 28 days of treatment, of significantly increasing the PCr/Ptotal ratio.

In the same time, while the PCr/ATP ratio measured on the “control” forearms decreased, that of the forearms treated by the formulation containing the Rhodiola crenulata extract increased. This difference was significant.

No improvement of the parameters measured was able to be observed on the forearms having received the placebo formulation, and this was the case whatever the measurement time was.

FIG. 2 represents the evolution of the PCr/Ptotal and PCr/ATP ratios (in %) with respect to D0 (day 0=day of application of the composition) after 28 days of application of a formulation containing 3% of Rhodiola crenulata.

In the Figure, the symbols used are:
•: significantly different from D0 (p<0.05)
ns: non-significantly different from D0
*: significantly different from the “control” group (p<0.05)

### FIG. 2 Values:

Evolution of the PCr/Ptotal ratio (in percentage) under “control” and “treated” conditions after 28 days:

| | Rhodiola crenulata | Control |
|---|---|---|
| Average | 3.72 | 0.05 |
| Standard deviation | 4.72 | 3.14 |

Evolution of the PCr/ATP ratio (in percentage) under “control” and “treated” conditions after 28 days:

TABLE IV

| | Rhodiola crenulata | Control |
|---|---|---|
| Average | 5.83 | −4.17 |
| Standard deviation | 10.56 | 4.92 |

The present study has enabled demonstrating that a formulation containing 3% (w/w) of a Rhodiola crenulata extract is capable in vivo of improving the energy metabolism of the cutaneous tissue. After 28 days of twice-daily applications, the results obtained do in fact show a significant effect of the product tested on the metabolism of phosphocreatine.

### Example 8

### Innocuity Tests

Evaluation of the cosmetic acceptation of a preparation containing the product of the invention.

Toxicology tests were carried out on the Rhodiola crenulata extract obtained according to Example 4, used pure, by an ocular evaluation in the rabbit, by the study of the absence of abnormal toxicity by single oral administration in the rat and by the study of the sensitizing power in the guinea pig.

### A) Evaluation of the Primary Irritation of the Skin in the Rabbit:

The preparations described above are applied without dilution at the dose of 0.5 ml on the skin of 3 rabbits according to the method recommended by the OECD in relation to the study of “the acute irritant/corrosive effect on the skin”.

The products are classed according to the criteria defined in the Decision of 1 Feb. 1982 published in the Official Journal of the French Republic of 21 Feb. 1982.

The results of these tests enabled concluding that the preparation retained was classed non-irritant for the skin.

### B) Evaluation of the Ocular Irritation in the Rabbit:

The preparations described above were instilled pure and in one batch at the rate of 0.1 ml in the eye of three rabbits of the method recommended by the OECD directive No. 405 of 24 Feb. 1987 in relation to the study of the “acute irritant/corrosive effect on the eyes”.

The results of this test enable concluding that the preparation can be considered as non-irritant for the eyes, in the sense of directive 91/326 EEC, used pure.

### C) Test on the Absence of Abnormal Toxicity by Single Oral Administration in the Rat:

The preparations described were administered in one batch orally at the dose of 5 g/Kg of body weight, to 5 male rats and 5 female rats according to a protocol inspired from the OECD directive No. 401 of 24 Feb. 1987 and adapted to cosmetic products.

The LD0 and LD50 are found to be greater than 5,000 mg/Kg. The preparation tested is therefore not classed amongst the preparations which are dangerous by ingestion.

### D) Evaluation of the Skin Sensitization Potential in the Guinea Pig:

The preparations described are subjected to the maximization test described by Magnusson and Kligmann, a protocol which is in agreement with the directive line No. 406 of the OECD.

The preparation is classed as non-sensitizing by contact with the skin.

In the following Examples, the term “products of the invention” represents the Rhodiola crenulata extracts according to the invention, and in particular according to Examples 1 to 4.

### Example 9

### “No Transfer” Lipstick

Use of the products of the invention in oil-in-water emulsion type cosmetic or pharmaceutical formulations.

### Formulation 9a:

| A | Water | qsp 100 |
|---|---|---|
| | Butylene Glycol | 2 |
| | Glycerine | 3 |
| | Sodium Dihydroxycetyl Phosphate, | 2 |
| | Isopropyl Hydroxycetyl Ether | |
| B | Glycol Stearate SE | 14 |
| | Triisononaoin | 5 |
| | Octyl Cocoate | 6 |
| C | Butylene Glycol, Methylparaben, | 2 |
| | Ethylparaben, Propylparaben, | |
| | pH adjusted to 5.5 | |
| D | Products of the invention | 0.01-10% |

### Formulation 9b:

| A | Water | qsp 100 |
|---|---|---|
| | Butylene Glycol | 2 |
| | Glycerine | 3 |
| | Polyacrylamide, Isoparaffin, | 2.8 |
| | Laureth-7 | |
| B | Butylene Glycol, Methylparaben, | 2 |
| | Ethylparaben, Propylparaben; | |
| | Phenoxyethanol, Methylparaben, | 2 |
| | Propylparaben, Butylparaben, | |
| | Ethylparaben | |
| | Butylene Glycol | 0.5 |
| D | Products of the invention | 0.01-10% |

### Formulation 9c:

| | | |
|---|---|---|
| A | Carbomer | 0.50 |
| | Propylene Glycol | 3 |
| | Glycerol | 5 |
| | Water | qsp 100 |
| B | Octyl Cocoate | 5 |
| | Bisabolol | 0.30 |
| | Dimethicone | 0.30 |
| C | Sodium Hydroxide | 1.60 |
| D | Phenoxyethanol, Methylparaben, | 0.50 |
| | Propylparaben, Butylparaben, | |
| | Ethylparaben | |
| E | Perfume | 0.30 |
| F | Products of the invention | 0.01-10% |

### Example 10

### Use of the Products of the Invention in a Water-in-Oil Tune Formulation

| | | |
|---|---|---|
| A | PEG 30 dipolyhydroxystearate | −3 |
| | Capric Triglycerides | 3 |
| | Cetearyl Octanoate | 4 |
| | Dibutyl Adipate | 3 |
| | Grape Seed Oil | 1.5 |
| | Jojoba Oil | 1.5 |
| | Phenoxyethanol, Methylparaben, | 0.5 |
| | Propylparaben, Butylparaben, | |
| | Ethylparaben | |
| B | Glycerine | 3 |
| | Butylene Glycol | 3 |
| | Magnesium Sulfate | 0.5 |
| | EDTA | 0.05 |
| | Water | qsp 100 |
| C | Cyclomethicone | 1 |
| | Dimethicone | 1 |
| D | Perfume | 0.3 |
| E | Products of the invention | 0.01-10% |

### Example 11

### Use of the Products of the Invention in a Shampoo or Shower Gel Type Formulation

| | | |
|---|---|---|
| A | Xanthan Gum | 0.8 |
| | Water | qsp 100 |
| B | Butylene Glycol, Methylparaben, | 0.5 |
| | Ethylparaben, Propylparaben | |
| | Phenoxyethanol, Methylparaben, | 0.5 |
| | Propylparaben, Butylparaben, | |
| | Ethylparaben | |
| C | Citric acid | 0.8 |
| D | Sodium Laureth Sulfate | 40.0 |
| E | Product of the invention | 0.01-10% |

### Example 12

### Use of the Products of the Invention in a Lipstick Type Formulation and Other Anhydrous Products

| | | |
|---|---|---|
| A | Mineral Wax | 17.0 |
| | Isostearyl Isostearate | 31.5 |
| | Propylene Glycol Dipelargonate | 2.6 |
| | Propylene Glycol Isostearate | 1.7 |
| | PEG 8 Beeswax | 3.0 |
| | Hydrogenated Palm Kernel Oil Glycerides, | 3.4 |
| | Hydrogenated Palm Glycerides | |
| | Lanolin Oil | 3.4 |
| | Sesame Oil | 1.7 |
| | Cetyl Lactate | 1.7 |
| | Mineral Oil, Lanolin Alcohol | 3.0 |
| B | Castor Oil | qsp 100 |
| | Titanium Dioxide | 3.9 |
| | CI 15850:1 | 0.616 |
| | CI 45410:1 | 0.256 |
| | CI 19140:1 | 0.048 |
| | CI 77491 | 2.048 |
| C | Products of the invention | 0.01-5% |

### Example 13

### Use of the Products of the Invention in an Aqueous Gel Formulation (Eyeliner, Slimming Products, Etc. . . . )

| A | Water | qsp 100 |
|---|---|---|
| | Carbomer | 0.5 |
| | Butylene Glycol | 15 |
| | Phenoxyethanol, Methylparaben, | 0.5 |
| | Propylparaben, Butylparaben, | |
| | Ethylparaben | |
| B | Products of the invention | 0.01-10% |

What is claimed is:

1. A method for increasing the energy metabolism of the cutaneous tissues of a subject comprising the topical application of a composition comprising a Rhodiola crenulata extract.

2. The method according to claim 1, wherein said cutaneous tissue is the skin.

3. The method according to claim 1, wherein said cutaneous tissue is selected from the group consisting of the epidermis, the dermis, and the dermis and the epidermis.

4. The method according to claim 1, for increasing the energy metabolism of cells selected from the group consisting of keratinocytes, fibroblasts, and keratinocytes and fibroblasts.

5. The method according to claim 1, wherein the subject is a mammal.

6. The method according to claim 1, wherein the topical composition comprises between 0.01% and 10% (w/w) of said Rhodiola crenulata extract.

7. The method according to claim 1, wherein the Rhodiola crenulata extract is obtained by extraction with a solvent selected from the group consisting of pentane, decane, cyclohexane, hexane, petroleum ether, monochloromethane, dichloromethane, chloroform, isopropanol, propanol, ethyl acetate, ethanol, methanol, acetone, butylene glycol, propylene glycol, pentylene glycol, glycerol, water, and any mixture of at least two of these solvents.

8. The method according to claim 7, wherein the solvent is a water-alcoholic or a water-glycolic mixture.

9. The method according to claim 1, wherein the extract is an extract selected from the group consisting of an extract of the roots of Rhodiola crenulata, an extract of the low stem of Rhodiola crenulata, and an extract of the roots and of the low stem of Rhodiola crenulata.

10. The method according to claim 1, wherein the extract is obtained by reduction in powder form of at least one part of the plant Rhodiola crenulata.

11. The method according to claim 1, wherein the extract contains a compound selected from the group consisting of crenulatine, and at least one of a crenulatine salt.

12. A method comprising the topical application of a composition comprising a Rhodiola crenulata extract for exerting an effect selected from the group consisting of fighting against the loosening of the cutaneous tissue, slowing down the loosening of the cutaneous tissue, increasing the plasticity of the cutaneous tissue, exerting an anti-wrinkle effect, slowing down the appearance of wrinkles, fighting against the appearance of acne, slowing down the appearance of acne, exerting a depigmenting effect of the cutaneous tissue, and promoting or peeling desquamation of the cutaneous tissue.

13. A method of cosmetic care, comprising topical application, on at least one area of the cutaneous tissue of a subject in need thereof, of the composition as defined in claim 1, for increasing the energy metabolism of at least said area of the cutaneous tissue of the subject.

14. The method of cosmetic care according to claim 13, wherein the composition comprises between 0.01% and 10% (w/w) of said Rhodiola crenulata extract.

15. The method according to claim 13, wherein the cosmetic care is selected from the group consisting of fighting against the loosening of the cutaneous tissue, slowing down the loosening of the cutaneous tissue, increasing the plasticity of the cutaneous tissue, exerting an anti-wrinkle effect, slowing down the appearance of wrinkles, fighting against the appearance of acne, slowing down the appearance of acne, exerting a depigmenting effect of the cutaneous tissue, and promoting desquamation or “peeling” of the cutaneous tissue.

16. The method according to claim 13, wherein increasing the capacity of the cells of the cutaneous tissue to multiply is increased.

17. A method of therapeutic treatment comprising topically applying, on at least one area of the cutaneous tissue of a subject, a therapeutically effective amount of a composition as defined in claim 1 for increasing the energy metabolism of at least said area of the cutaneous tissue of the subject.

18. A method of treatment according to claim 17 wherein the treatment is selected from the group consisting of fighting against the loosening of the cutaneous tissue, slowing down the loosening of the cutaneous tissue, increasing the plasticity of the cutaneous tissue, exerting an anti-wrinkle effect, slowing down the appearance of wrinkles, fighting against the appearance of acne, slowing down the appearance of acne, exerting a depigmenting effect of the cutaneous tissue, and promoting desquamation or “peeling” of the cutaneous tissue.

19. A method of treatment according to claim 17 wherein the capacity of the cells of the cutaneous tissue to multiply is increased.
